(19) United States
(12) Patent Application Publication
(10) Pub. No.: US 2026/0363678 A1
(43) Pub. Date: Nov. 19, 2026

(54) Throat Specimen Collection Devices with a Bite Element for Positioning the Collection Receptacle

(71) Applicant: Abbott Diagnostics Scarborough, Inc., Scarborough, ME (US)

(72) Inventors: Victor Shapiro, Scarborough, ME (US); Bryan Johnson, San Diego, CA (US); Bryce Gibson, Scarborough, ME (US)

(21) Appl. No.: 19/133,461

(22) PCT Filed: Dec. 1, 2023
(86) PCT No.: PCT/US2023/082180

§ 371 Date: May 28, 2025

Related U.S. Application Data
(60) Provisional application No. 63/429,353, filed on Dec. 1, 2022.

(51) Int. Cl. A61B 10/02; A61B 5/00; A61F 13/38

(52) U.S. Cl. CPC A61B 10/02; A61B 5/682; A61F 13/38; A61B 2010/0216

(57) ABSTRACT

Provided are throat specimen collection devices and methods of using them. In certain embodiments, a throat specimen collection device comprises: i) a bite element, and ii) a collection receptacle comprising an elongated member and a collection member, wherein, when the subject bites down on the bite element to restrain the element within the subject's mouth and the collection receptacle is inserted into the throat to a collection position, the collection member touches the throat of the subject and collects the throat specimen from the subject. Methods of collecting throat specimens are also provided, the methods comprising, employing a throat specimen collection device to a subject's mouth and collecting a throat specimen.

### CROSS-REFERENCE TO RELATED APPLICATIONS

This application claims the benefit of U.S. Provisional Patent Application No. 63/429,353, filed Dec. 1, 2022, which application is incorporated herein by reference in its entirety.

### INTRODUCTION

Collecting throat specimens typically involves a health-care professional to introduce a throat specimen collection apparatus into a subject's mouth. Throat specimen collection apparatuses are generally elongated structures with a throat specimen collection receptacle at the end. A health-care professional introduces the throat specimen collection apparatus into a subject's open mouth and touches the throat with the collection receptacle.

These devices and methods are often unpleasant for the subjects. For example, certain patients, particularly, children exhibit anxiety with the collection devices and procedures. Certain patients may experience unpleasant gagging during the collection. Therefore, collecting throat specimens from subjects is often challenging and unpleasant for both the subjects as well as the health-care professionals.

### SUMMARY

The disclosure provides throat specimen collection devices and methods of collecting throat specimens using such devices. The throat specimen collection devices disclosed herein address the problems discussed above with prior devices and collection methods.

The throat specimen collection devices and methods disclosed herein can be used to avoid some of the unpleasant experience in throat specimen collection. For example, the throat specimen collection devices and methods disclosed herein, when used with children may reduce the child's anxiety during the throat specimen collection. Also, the throat specimen collection devices and methods disclosed herein may avoid unpleasant gagging during collection in certain subjects.

In certain embodiments, a throat specimen collection device comprises:
i) a bite element, and
ii) a collection receptacle comprising, an elongated member and a collection member
wherein, when the subject bites down on the bite element to restrain the element within the subject's mouth and the collection receptacle is inserted into the throat to a collection position, the collection receptacle touches the throat of the subject and collects the throat specimen from the subject.

In some cases, the collection receptacle is movable between an introduction position and the collection position. The introduction position of the collection receptacle is the position in which the collection receptacle does not touch the throat of the subject, for example, when the collection receptacle is introduced into the bite element. The collection receptacle while in the introduction position can be moved into the collection position, where the collection member touches the throat of a subject.

The collection receptacle may comprise an elongated member having at one end a collection member. The collection member may be a cotton swab or a brush head, such as a nubby silicon brush head. The collection member may also be a rayon swab, polyester swab, foam swab, or flocked swab.

The bite element can be configured to maintain itself within a subject's mouth when the subject bites down on the bite element using the subject's anterior teeth. In certain devices, when the subject bites down on the bite element, the bite element restrains the subject's tongue as well. In such embodiments, the bite element can be popsicle shaped to fit in the subject's mouth, particularly, over the subject's tongue. The bite element can be made of any suitable material, such as wood, plastic, rubber, cardboard paper, or silicon. The bite element can also be flavored.

When the subject bites down on the bite element, the collection receptacle can be moved into the collection position where the collection member touches the subject's throat. Alternatively, when the subject bites on the bite element, the collection receptacle can be in an introduction position where the collection member does not touch the subject's throat. The collection receptacle can then be moved from the introduction position to the collection position. For example, the collection receptacle in the introduction position can be moved through the bite element into the collection position.

Also provided are methods of collecting throat specimens using the throat specimen collection devices. Such methods comprise:
i) the subject biting down on the bite element of the throat specimen collection device to restrain the bite element in the subject's mouth,
ii) collecting the throat specimen of the subject when the collection receptacle is in the collection position.

In certain cases, the method comprises moving the throat specimen collection receptacle from an introduction position to the collection position.

### BRIEF DESCRIPTION OF THE DRAWINGS

FIGS. 1A-1C show an embodiment of a throat specimen collection device.

FIGS. 2A-2B show an embodiment of a throat specimen collection device.

FIGS. 3A-3B show another embodiment of throat specimen collection device.

FIGS. 4A-4B show a further embodiment of a throat specimen collection device.

FIG. 5 shows an example of a nubby silicon brush head that can be used as a collection member.

### DETAILED DESCRIPTION

Provided are throat specimen collection devices and methods of collecting throat specimen. In certain embodiments, a throat specimen collection device comprises:
i) a bite element, and ii) a collection receptacle comprising, an elongated member and a collection member,
wherein, when the subject bites down on the bite element to restrain the element and the collection receptacle is in the collection position, the collection member touches the throat of the subject and collects the throat specimen from the subject.

Also provided are methods of collecting throat specimens using the throat specimen collection devices disclosed herein. The methods comprise:
i) the subject biting down on the bite element of the throat specimen collection device to restrain the bite element in the subject's mouth, and
ii) collecting the throat specimen of the subject when the collection receptacle is in the collection position.

Before the present disclosure is described in greater detail, it is to be understood that this disclosure is not limited to any particular embodiments described. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the disclosure. The upper and lower limits of these smaller ranges may independently be included or excluded in the range, and each range where either, neither or both limits are included in the smaller ranges is also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, some potential and exemplary methods and materials may now be described. Any and all publications mentioned herein are incorporated by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a,” “an,” and “the” include plural referents unless the context clearly dictates otherwise. Thus, for example, reference to “a device” includes a plurality of such devices. It is further noted that the claims may be drafted to exclude any element, e.g., any optional element. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Further, the dates of publication provided may be different from the actual publication dates which may need to be independently confirmed. To the extent the definition or usage of any term herein conflicts with a definition or usage of a term in an application or reference incorporated by reference herein, the instant application shall control.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order which is logically possible.

### Definitions

As used herein, the term “a throat specimen” refers to a biological sample obtained from the back of a subject's mouth from the area at the top part of the tubes that go down into the subject's stomach and lungs. Throat specimens typically comprise microbes from the throat of a subject and may also contain the subject's cells, mucus, saliva, and other secretions. The term specimen may be used interchangeably with “throat sample” and retain the same meaning.

A “swab” is an absorbent pad or piece of material used for collecting specimens.

A “bite element” refers to a part of the throat specimen collection device that is configured to allow a subject to bite and restrain the bite element in the subject's mouth so that the bite element is substantially immobile in the subject's mouth.

A “collection member” as used herein refers to a part of the collection receptacle that is configured to obtain a specimen from a subject's throat. The collection receptacle comprises an elongated member having at one end a collection member which can be a swab, for obtaining a specimen from a subject's throat.

The term “anterior teeth” refers to the teeth in the front of a subject's mouth. These teeth typically comprise incisors and canines.

### Throat Specimen Collection Devices

As described above, the throat specimen collection devices, in certain embodiments, comprise:
i) a bite element, and
ii) a collection receptacle comprising, an elongated member and a collection member
wherein, when the subject bites down on the bite element to restrain the bite element and the collection receptacle is in the collection position, the collection member touches the throat of the subject and collects the throat specimen from the subject.

The collection receptacle can be a physically integrated element of the throat specimen collection device. Alternatively, the collection receptacle can be a physically separate unit but can be physically fitted into the bite element to constitute a throat specimen collection device.

In some cases, when the subject bites down on the bite element, the collection receptacle is in the collection position. While in the collection position, the collection receptacle is positioned such that the collection member touches the throat of the subject and collects the throat specimen from the subject.

In some cases, the collection receptacle is movable between an introduction position and the collection position. The introduction position of the collection receptacle is the position in which the collection receptacle does not touch the throat of the subject, for example, when the collection receptacle is introduced into the bite element. The collection receptacle while in the introduction position can be moved into the collection position, where the collection member touches the throat of a subject. In some cases, the collection receptacle while in the introduction position is slidable through the bite element into the collection position.

In some cases, when a subject bites down on the bite element, the collection receptacle is in the introduction position, where the collection receptacle does not touch the throat of a subject. The collection receptacle while in an introduction position can be moved into the collection position, where the collection receptacle touches the throat of a subject and collects the throat specimen from the subject.

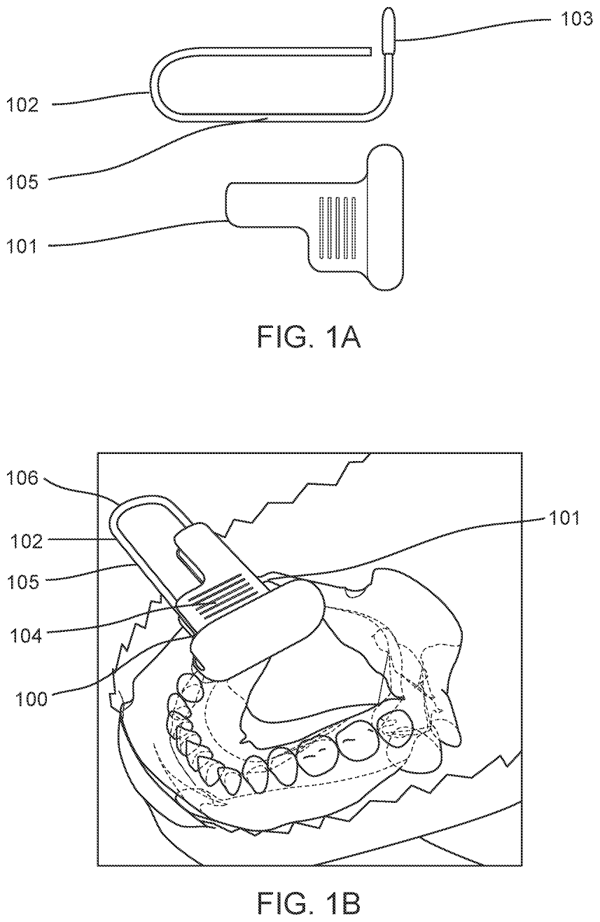

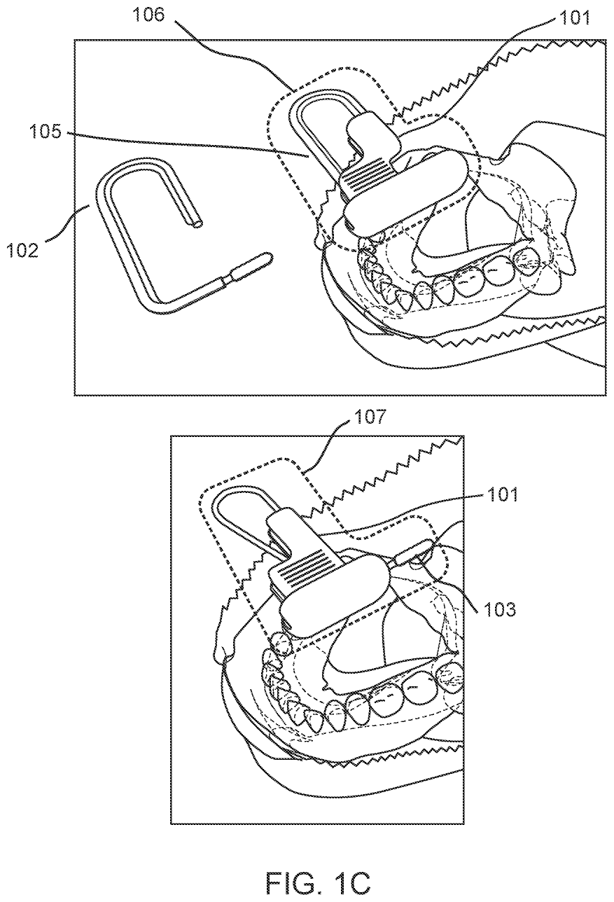

A non-limiting example of this embodiment is provided in FIGS. 1A-1C.

FIG. 1A shows an exemplary embodiment of a throat specimen collection device. This device comprises the bite element 101 and the throat specimen collection receptacle 102. The throat collection receptacle comprises the longitudinal rod 105 and the collection member 103.

As shown in FIG. 1B, in this embodiment, when the throat specimen collection device 100 is placed into a subject's mouth, the subject can bite down on the bite element 101 using the subject's pre-molars and/or molars to restrain the bite element in the subject's mouth. The bite element 101 can have a rough surface 104 to provide the subject a stronger grip on the bite element compared to a smooth surface. The rough surface 104 can have ridges, raised dots, depressed dots, waves, or other suitable pattern that would provide the subject a stronger grip on the bite element.

As shown in FIG. 1B and top panel of FIG. 1C, the throat specimen collection receptacle 102 is in an introduction position 106, indicated by the dotted line in FIG. 1C. Thus, the introduction position 106 encompasses any position of the collection receptacle where the collection member does not touch the subject's throat, i.e., not in a collection position. In the top panel of FIG. 1C, the collection member 103 (not shown), is hidden within the bite element 101. The collection member 103 does not touch the throat of the subject while in the introduction position. A health-care professional can move the collection receptacle from the introduction position 106 (FIG. 1C, top panel, dotted line) into the collection position 107 (FIG. 1C, bottom panel, dotted line) by pushing on the longitudinal rod 105 which in turn pushes the throat specimen collection receptacle 103 towards the subject's throat and into the throat specimen collection position 107. While in the collection position 107, the collection member 103 touches the throat of a subject and collects the throat specimen from the subject, as shown in the bottom panel of FIG. 1C.

In cases where the subject bites on the bite element using the pre-molars and/or molars to restrain the bite element, the introduction position of the collection receptacle can be towards the subject's cheek. Thus, the collection receptacle can be introduced into the subject's mouth near the subject's cheek and the collection receptacle can then be moved into the subject's mouth such that collection member touches the subject's throat.

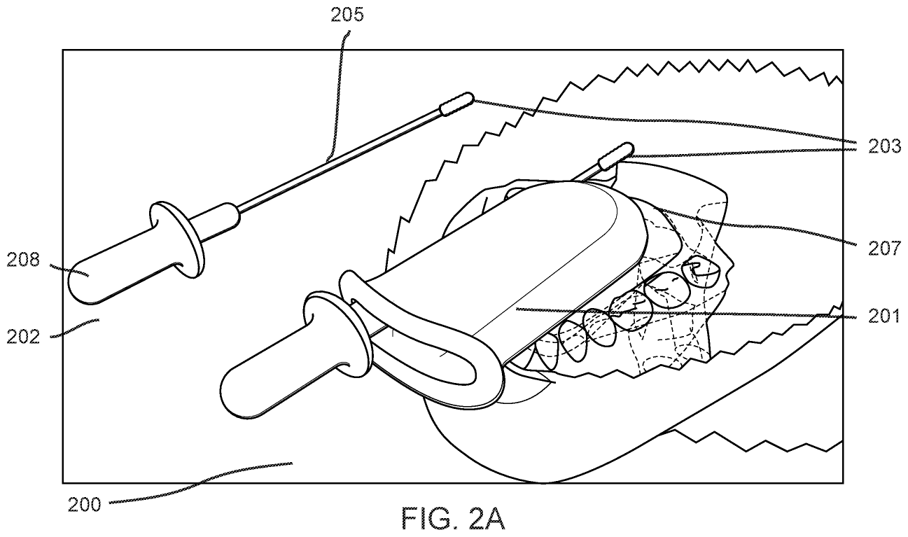

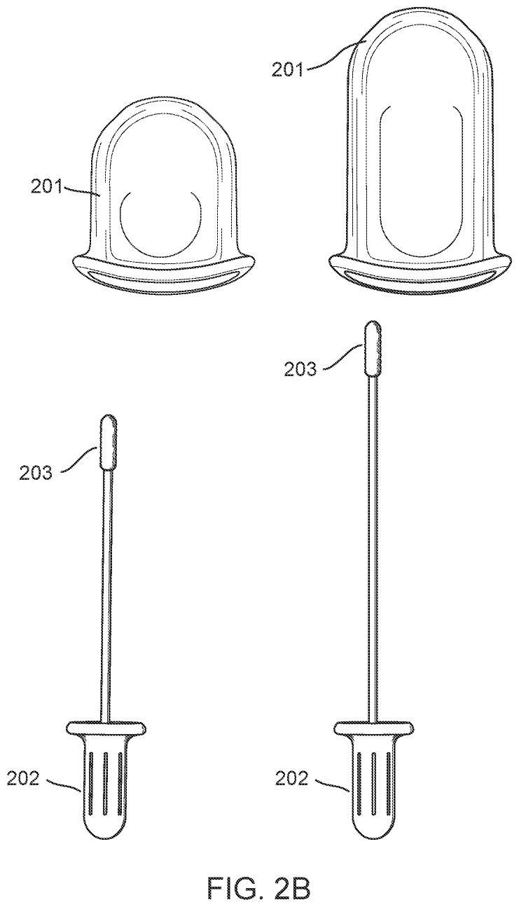

In some cases, a collection receptacle is physically separate from the device, for example, as shown in FIG. 1A and FIG. 2A. When the subject bites on the bite element, the collection receptacle can be introduced into the device at the introduction position, where the collection member does not touch the throat of a subject. The collection receptacle in the introduction position can then be moved into the collection position, where the collection member touches the throat of a subject and collects the throat specimen from the subject.

For example, as shown in the top panel of FIG. 1C, the collection receptacle 102, which is separate from the bite element 101 can be introduced into the introduction position 106 after the subject restrains the bite element by biting down on it. A health-care professional, can move the collection receptacle from the introduction position into the collection position by pushing on the longitudinal rod 105 which in turn pushes the collection member 103 towards the subject's throat and into the collection position 107. While in the collection position 107, the collection member 103 touches the throat of a subject and collects the throat specimen from the subject, as shown in FIG. 1C.

Another non-limiting example in which the bite element and the collection receptacle are physically separate is also provided in FIGS. 2A-2B as throat specimen collection device 200. The collection receptacle 202 comprises the holder 208, the longitudinal member 205, and the collection member 203. The collection receptacle can be physically fitted into the bite element to constitute the throat specimen collection device. In this embodiment, a subject bites down on the bite element 201 using the subject's anterior teeth to restrain the bite element 201 in the subject's mouth. The collection receptacle 202, which is physically separate from the bite element 201, can be fitted into an introduction position (not shown) where the collection member does not touch the throat of the subject. A health-care professional, can then move the collection receptacle from the introduction position (not shown) into the collection position 207, where the collection member 203 touches the throat of a subject and collects the throat specimen from the subject, as shown in FIG. 2A.

The size of the bite element 201 can be varied depending on the size of a subject's mouth. For example, as shown in FIG. 2B, a smaller bite element 201 (left) can be used for a child whereas a bite element 201 (right) can be used for an adult.

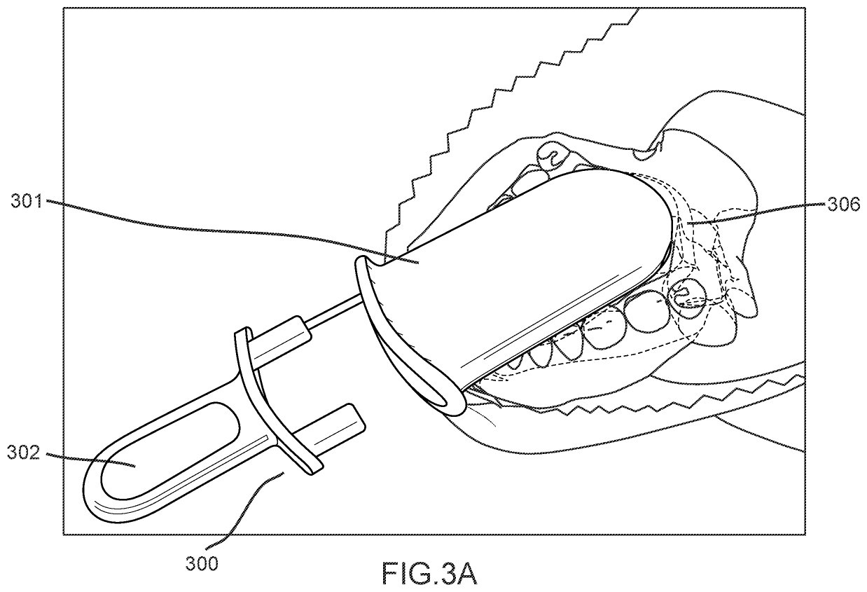

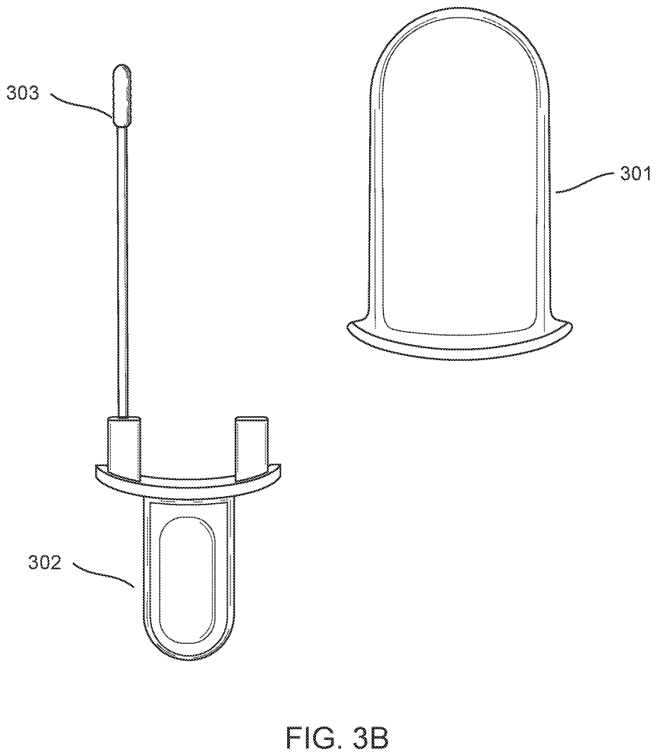

Another non-limiting example of the throat specimen collection device 300 where a collection receptacle 302 is physically separate from the bite element 301 is provided in FIGS. 3A-3B. In this embodiment, a subject bites down on the bite element 301 using the subject's anterior teeth to restrain the bite element in the subject's mouth. The collection receptacle 302, which is separate from the device, can be inserted into an introduction position 306 where the collection member 303, which is hidden within the bite element 301, does not touch the throat of the subject. A health-care professional can then move the throat specimen collection receptacle from the introduction position into the collection position (not shown), where the collection member 303 touches the throat of a subject and collects the throat specimen.

In cases where a subject bites down on the bite element using the subject's anterior teeth to restrain the bite element in the subject's mouth, the introduction position of the throat specimen collection receptacle can be towards the subject's lips. Thus, the collection receptacle can be introduced into the subject's mouth near the subject's lips and the collection receptacle can then be moved into the subject's mouth such that collection member touches the subject's throat.

In some cases, a collection receptacle can contain more than one collection members. For example, as shown in FIGS. 3A-3B, the collection receptacle contains two locations where an elongated member can be loaded containing a throat specimen collection receptable. While two such receptacles are not shown in FIGS. 3A and 3B, it is readily apparent from these figures that the throat specimen collection receptacle shown in FIGS. 3A and 3B can contain two elongated members, each having a collection member.

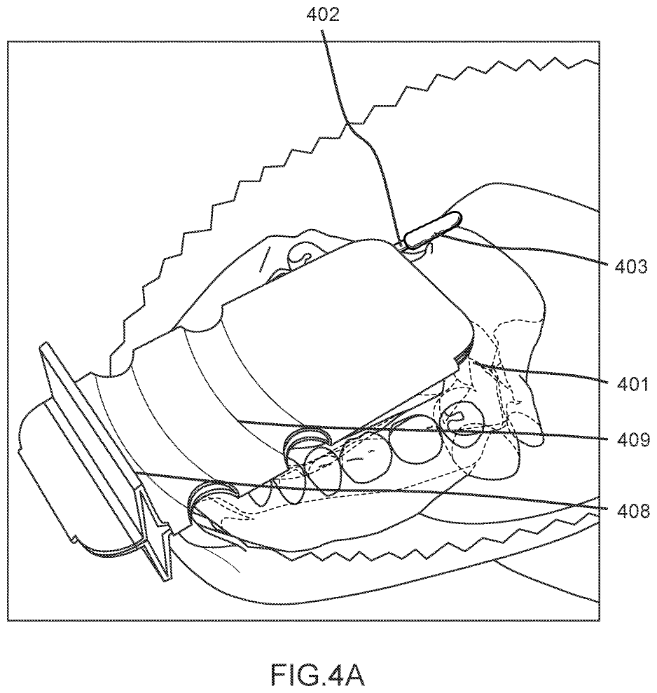

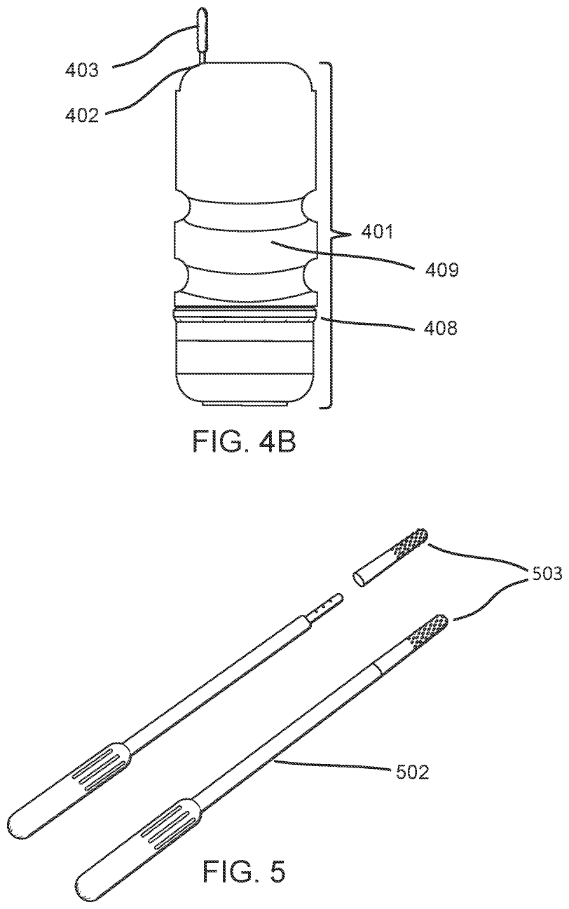

In some cases, the bite element can contain a means to modulate the length of the bite element, such that the bite element can be elongated or shortened based on the size of a subject's mouth. For example, the bite element 401 can comprise a hinge 408, as shown in FIGS. 4A-4B. Alternatively, a bite element can have a serrated surface 409 that can extend or contract to modulate the length of the bite element. The bite element 401 can restrain the subject's tongue. The throat specimen collection device further comprises the throat specimen collection receptacle comprising the elongated member 402 and the collection member 403.

In some cases, the collection receptacle is an elongated member 502 having at one end the collection member 503 for collecting the throat specimen. The collection member can be a cotton swab or a brush head, such as a nubby silicon brush head as shown in FIG. 5. The collection member can also be a rayon swab, polyester swab, foam swab, or flocked swab.

In some cases, when a subject bites down on the bite element to restrain it, the bite element restrains the subject's tongue. In certain such cases, the bite element is popsicle shaped, which covers a subject's tongue to restrain the tongue.

The bite element can be made of any suitable material, for example, a cardboard paper, rubber, plastic, or silicone.

Because the bite element can touch a subject's tongue, the bite element can be configured to avoid unpleasant taste/flavor and/or to provide a pleasant taste/flavor. For example, the bite element can be flavored by incorporating flavored substances into the materials used to produce the bite element. When the bite element touches a subject's tongue, such flavored material can dissolve into the subject's saliva and provide a pleasant taste to the subject. In particular, the bite element with a pleasant taste would facilitate obtaining throat specimens from children as children may be distracted from the unpleasant experience of taking the throat specimen and instead enjoy the pleasant taste.

In some cases, the bite element is marked to indicate the appropriate location for the subject's bite. An appropriate location for the bite element is such that when a subject restrains the bite element, the bite element is properly placed in the subject's mouth so that the collection member can properly touch the throat and collect a throat specimen from the subject. A marking on a bite element shows the appropriate location for the subject's bite to avoid the situations where: 1) the collection receptacle does not reach the subject's throat thereby failing to obtain a throat specimen or 2) the collection receptacle is too close to the subject's throat thereby possibly causing injury and/or pain.

### Methods of Collecting Throat Specimens

Certain embodiments provide a method comprising:
i) the subject biting down on the bite element of the throat specimen collection device to restrain the bite element in the subject's mouth, and
ii) collecting the throat specimen of the subject when the collection member is in the collection position.

In some embodiments of the devices, when a subject bites down on a bite element to restrain the bite element in the subject's mouth, the collection receptacle is in the collection position. In this position the collection member of the collection receptacle touches the throat of the subject and collects the throat specimen from the subject. Thus, when using such devices, the methods comprise i) The subject biting down on the bite element of the throat specimen collection device to restrain the bite element in the subject's mouth, and ii) collecting the throat specimen of the subject when the collection member receptacle is in the collection position.

Also, in some embodiments of the devices, when a subject bites down on a bite element, the collection receptacle is in the introduction position, where the collection member does not touch the throat of a subject. The collection receptacle while in the introduction position can then be moved into the collection position, where the collection member touches the throat of a subject and collects the throat specimen from the subject. Thus, when using such devices, the methods disclosed herein comprise: i) the subject biting down on the bite element of the throat specimen collection device to restrain the bite element in the subject's mouth, and ii) moving the collection member from an introduction position to a collection position thereby collecting the throat specimen of the subject.

Moreover, in some cases, the collection receptacle is a physically separate unit but can be physically fitted within the bite element. In these cases, when the subject bites down on the bite element, the collection receptacle can be fitted into the device at an introduction position, where the collection member does not touch the throat of a subject. The collection receptacle in an introduction position can then be moved into the collection position, where the collection member touches the throat of the subject and collects the throat specimen from the subject. Thus, when using such devices, the methods disclosed comprise: i) the subject biting down on the bite element of the throat specimen collection device to restrain the bite element in the subject's mouth, ii) introducing a collection receptacle into the introduction position, and iii) moving the collection member from the introduction position into the collection position thereby collecting the throat specimen of the subject.

When the collection receptacle is a physically separate but can be physically fitted within the bite element, the collection receptacle can be physically fitted within the bite element before placing the device in a subject's mouth. In such cases, the method comprises: i) physically fitting the collection receptacle into the bite element, ii) the subject biting down on the bite element of the throat specimen collection device to restrain the bite element in the subject's mouth, and iii) collecting the throat specimen of the subject.

The throat specimen can be collected if the collection receptacle within the bite element is in a position where, when the subject bites on the bite element, the collection member touches the throat of the subject. Alternatively, where the subject bites on the bite element and the collection member does not touch the throat of the subject, the collection receptacle can be moved from an introduction position to the collection position thereby collecting the throat specimen of the subject.

### Utility

The throat specimen collection devices and methods of collecting throat specimen as disclosed herein can be typically used in medical testing and diagnostics. As appreciated by a person of ordinary skill in the art, a throat specimen from a subject can be tested for infections, such as bacterial or viral infections. A throat specimen from a subject can be tested for collecting cells from a subject for DNA based diagnostics. Cells collected from a subject can also be tested for the presence of neoplasm.

In some cases, a throat specimen from a subject can be tested for detecting an infection with a pathogen, for example, a virus, a bacterium, or a fungus.

A pathogen that can be tested with a throat specimen includes one or more of human immunodeficiency virus (HIV), Hepatitis C virus (HCV), Hepatitis B virus (HBV), Chlamydia trachomatis (CT), Neisseria gonorrhoeae (NG), Human papillomavirus (HPV), Cytomegalovirus (CMV), Epstein-Barr virus (EBV), Polyomavirus BK (BKV), Methicillin-resistant Staphylococcus aureus (MRSA), Clostridium difficile (C. Diff.), Vancomycin-resistant enterococci (VRE), adenovirus, Mycobacterium tuberculosis (TB), Varicella Zoster Virus (VZV), Herpes simplex virus (HSV), John Cunningham virus (JCV), enterovirus, Lymphogranuloma Venereum (LGV), viruses of a Respiratory Viral Panel (RVP), Human Herpesvirus 6 (HHV6), Trichomonas vaginalis, Mycoplasma genitalium, norovirus, zika virus, Respiratory syncytial virus (RSV), streptococcal pathogen, such as Streptococcus pyrogenes and Streptococcus agalactiae, SARS-CoV-2, and influenza virus.

A throat specimen from a subject can also be tested for toxicological screening, such as for detecting drugs or other chemicals. A drug or compound tested in a throat specimen includes alcohol, amphetamines, barbiturates, benzodiazepines, cocaine, marijuana, methamphetamine, opioids, opiates, and phencyclidine.

For reasons of completeness, various aspects of the invention are set out in the following numbered clauses:
Clause 1. A throat specimen collection device comprising:
i) a bite element,
ii) a collection receptacle comprising, an elongated member and a collection member
wherein, when the subject bites down on the bite element to restrain the element within the subject's mouth and the collection receptacle is inserted into the throat to a collection position, the collection member touches the throat of the subject and collects the throat specimen from the subject.
Clause 2. The throat specimen collection device of clause 1, wherein the bite element is configured to allow a subject to bite on the bite element to restrain the bite element in the subject's mouth.
Clause 3. The throat specimen collection device of clause 2, wherein, when the subject bites on the bite element to restrain the bite element in the subject's mouth and the collection receptacle is in the collection position, the collection receptacle touches the throat of the subject and collects the throat specimen from the subject.
Clause 4. The throat specimen collection device of any one of clauses 1 to 3, wherein the collection receptacle can fit within the device such that when the subject bites on the bite element, the collection receptacle is in the collection position.
Clause 5. The throat specimen collection device of any one of clauses 1 to 4, wherein the collection receptacle is movable between an introduction position and the collection position.
Clause 6. The throat specimen collection device of clause 5, wherein the collection receptacle while in the introduction position is slidable through the bite element into the collection position.
Clause 7. The throat specimen collection device of any one of clauses 1 to 6, wherein the collection receptacle is an elongated member having at one end the collection member.
Clause 8. The throat specimen collection device of any one of clauses 1 to 7, wherein the collection member is a cotton swab, brush head, rayon swab, polyester swab, foam swab, or flocked swab.
Clause 9. The throat specimen collection device of clause 8, wherein the brush head of the collection member is a nubby silicon brush head.
Clause 10. The throat specimen collection device of any one of clauses 1 to 9, wherein the bite element is configured such that to restrain the bite element in the subject's mouth when the subject bites down on the bite element using the subject's anterior teeth.
Clause 11. The throat specimen collection device of clause 10, wherein when the subject bites down on the bite element, the bite element restrains the subject's tongue.
Clause 12. The throat specimen collection device of any one of clauses 1 to 3, wherein the bite element is popsicle shaped.
Clause 13. The throat specimen collection device of any one of clauses 1 to 3, wherein the bite element is made of a cardboard paper.
Clause 14. The throat specimen collection device of any one of clauses 1 to 3, wherein the bite element is made of silicone.
Clause 15. The throat specimen collection device of any one of clauses 1 to 3, wherein the bite element is flavored.
Clause 16. The throat specimen collection device of any one of clauses 1 to 3, wherein the bite element is marked to indicate the appropriate location for the subject's bite down on the bite.
Clause 17. The throat specimen collection device of clause 5, wherein the introduction position of the collection receptacle is towards the subject's lips when the subject bites on the bite element using the subject's anterior teeth to restrain the bite element.
Clause 18. The throat specimen collection device of any one of clauses 1 to 3, wherein the bite element is configured such that to restrain the bite element in the subject's mouth the subject bites on the bite element using the subject's pre-molars and/or molars.
Clause 19. The throat specimen collection device of clause 18, wherein the collection receptacle introduction position is near the subject's cheek when the subject bites on the bite element using the subject's pre-molars and/or molars to restrain the bite element.
Clause 20. A method of collecting a throat specimen of a subject using a throat specimen collection device, comprising:
i) the subject biting down on the bite element of the throat specimen collection device to restrain the bite element in the subject's mouth,
ii) collecting the throat specimen of the subject when the collection receptacle is in the collection position.
Clause 21. The method of clause 20, further comprising moving the collection receptacle from an introduction position to the collection position.
Clause 22. The method of clause 21, further comprising introducing the collection receptacle at the introduction position before moving the collection receptacle from the introduction position to the collection position.
Clause 23. The method of clause 20, further comprising collecting the collection member to obtain the throat specimen from the collection receptacle.

Although the foregoing disclosure has been described in some detail by way of illustration and example for purposes of clarity of understanding, it is readily apparent to those of ordinary skill in the art and in light of the teachings of this disclosure that certain changes and modifications may be made thereto without departing from the spirit or scope of the appended claims.

Accordingly, the preceding merely illustrates the principles of the disclosure. It will be appreciated that those skilled in the art will be able to devise various arrangements which, although not explicitly described or shown herein, embody the principles of the disclosure and are included within its spirit and scope. Furthermore, all examples and conditional language recited herein are principally intended to aid the reader in understanding the principles of the disclosure and the concepts contributed by the inventors to furthering the art, and are to be construed as being without limitation to such specifically recited examples and conditions. Moreover, all statements herein reciting principles, aspects, and embodiments of the disclosure as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present disclosure, therefore, is not intended to be limited to the exemplary embodiments shown and described herein. Rather, the scope and spirit of present disclosure is embodied by the appended claims. In the claims, 35 U.S.C. § 112(f) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112(f) is not invoked.

We claim:

1. A throat specimen collection device comprising:
i) a bite element,
ii) a collection receptacle comprising, an elongated member and a collection member
wherein, when the subject bites down on the bite element to restrain the element within the subject's mouth and the collection receptacle is inserted into the throat to a collection position, the collection member touches the throat of the subject and collects the throat specimen from the subject.

2. The throat specimen collection device of claim 1, wherein the bite element is configured to allow a subject to bite on the bite element to restrain the bite element in the subject's mouth.

3. The throat specimen collection device of claim 2, wherein, when the subject bites on the bite element to restrain the bite element in the subject's mouth and the collection receptacle is in the collection position, the collection receptacle touches the throat of the subject and collects the throat specimen from the subject.

4. The throat specimen collection device of any one of claims 1 to 3, wherein the collection receptacle can fit within the device such that when the subject bites on the bite element, the collection receptacle is in the collection position.

5. The throat specimen collection device of any one of claims 1 to 4, wherein the collection receptacle is movable between an introduction position and the collection position.

6. The throat specimen collection device of claim 5, wherein the collection receptacle while in the introduction position is slidable through the bite element into the collection position.

7. The throat specimen collection device of any one of claims 1 to 6, wherein the collection receptacle is an elongated member having at one end the collection member.

8. The throat specimen collection device of any one of claims 1 to 7, wherein the collection member is a cotton swab, brush head, rayon swab, polyester swab, foam swab, or flocked swab.

9. The throat specimen collection device of claim 8, wherein the brush head of the collection member is a nubby silicon brush head.

10. The throat specimen collection device of any one of claims 1 to 9, wherein the bite element is configured such that to restrain the bite element in the subject's mouth when the subject bites down on the bite element using the subject's anterior teeth.

11. The throat specimen collection device of claim 10, wherein when the subject bites down on the bite element, the bite element restrains the subject's tongue.

12. The throat specimen collection device of any one of claims 1 to 3, wherein the bite element is popsicle shaped.

13. The throat specimen collection device of any one of claims 1 to 3, wherein the bite element is made of a cardboard paper.

14. The throat specimen collection device of any one of claims 1 to 3, wherein the bite element is made of silicone.

15. The throat specimen collection device of any one of claims 1 to 3, wherein the bite element is flavored.

16. The throat specimen collection device of any one of claims 1 to 3, wherein the bite element is marked to indicate the appropriate location for the subject's bite down on the bite.

17. The throat specimen collection device of claim 5, wherein the introduction position of the collection receptacle is towards the subject's lips when the subject bites on the bite element using the subject's anterior teeth to restrain the bite element.

18. The throat specimen collection device of any one of claims 1 to 3, wherein the bite element is configured such that to restrain the bite element in the subject's mouth the subject bites on the bite element using the subject's pre-molars and/or molars.

19. The throat specimen collection device of claim 18, wherein the collection receptacle introduction position is near the subject's cheek when the subject bites on the bite element using the subject's pre-molars and/or molars to restrain the bite element.

20. A method of collecting a throat specimen of a subject using a throat specimen collection device, comprising:
i) the subject biting down on the bite element of the throat specimen collection device to restrain the bite element in the subject's mouth,
ii) collecting the throat specimen of the subject when the collection receptacle is in the collection position.

21. The method of claim 20, further comprising moving the collection receptacle from an introduction position to the collection position.

22. The method of claim 21, further comprising introducing the collection receptacle at the introduction position before moving the collection receptacle from the introduction position to the collection position.

23. The method of claim 20, further comprising collecting the collection member to obtain the throat specimen from the collection receptacle.
